(19) United States
(12) Patent Application Publication
(10) Pub. No.: US 2024/0366701 A1
(43) Pub. Date: Nov. 7, 2024

(54) EXTRACT OF MYROTHAMNUS SP FOR PROMOTING HAIR GROWTH

(71) Applicant: Lubrizol Advanced Materials, Inc., Cleveland, OH (US)

(72) Inventors: Cristina BONELL, Barcelona (ES); Núria ALMIÑANA DOMÈNECH, Barcelona (ES); Sylesh Kumar VENKATARAMAN, Frisco, TX (US); Rebecca CHISHOLM, Grapevine, TX (US)

(73) Assignee: Lubrizol Advanced Materials, Inc., Cleveland, OH (US)

(21) Appl. No.: 18/687,081

(22) PCT Filed: Aug. 23, 2022
(86) PCT No.: PCT/IB2022/057886

§ 371 Date: Feb. 27, 2024

(30) Foreign Application Priority Data
Sep. 3, 2021 (EP) 21382795.9

(51) Int. Cl. A61K 36/185; A61P 17/14

(52) U.S. Cl. CPC A61K 36/185; A61P 17/14; A61K 2236/17; A61K 2236/35; A61K 2236/37

(57) ABSTRACT

A botanical extract of Myrothamnus sp. is used in a method for one or more of: promoting hair growth, increasing hair growth, delaying hair loss, hindering hair loss, and increasing the thickness of hair of a subject, in particular the hair of the eyelashes and/or eyebrows. The method includes administering the botanical extract of Myrothamnus sp. to the subject. The extract is one which is obtained using subcritical water as an extraction solvent. Methods of preparing the botanical extract and compositions comprising the extract are also disclosed.

This application claims the priority of International Application PCT/IB2022/057886, filed Aug. 23, 2022, and EP21382795.9, filed Sep. 3, 2021, from which the PCT application claims priority, the disclosures of which are incorporated herein in their entireties by reference.

### FIELD OF THE INVENTION

The invention relates to the use of a botanical extract of Myrothamnus sp. for promoting hair growth, and/or preventing hair loss, and/or increasing the thickness of hair. In one embodiment, the hair is the hair of the eyebrows and/or the eyelashes. Methods of preparing the botanical extract and compositions comprising same are also disclosed.

### BACKGROUND OF THE INVENTION

Hair, and, in particular, the hair of the eyelashes and eyebrows, plays an important role in the perception of beauty in humans. Eyelashes and eyebrows are prominent features on a face. Eyelash and eyebrow hairs serve to protect the eyes and influence facial expressions. These hairs grow relatively slowly and have a short average length. In many cultures, long and thick eyelashes are particularly prized and sought after. There is an interest in enhancing the look of one's eyelashes by increasing the length and the thickness of the eyelashes. Further, there is an interest in enhancing the look of one's eyebrows by increasing the thickness of the eyebrows.

Hair damage and/or loss are common problems in human subjects and can have a great impact on a person's overall appearance. The hairs of the eyelashes and the eyebrows are particularly prone to damage due to touching by the fingers/hands, for example, when a subject rubs their eyes with their hands when they are tired. Mechanical or chemical stress during styling can weaken and damage hair. Hair loss can have other numerous causes (e.g., genetic disposition, aging and/or disease). There is considerable interest in treatments that can increase hair strength, prevent hair loss or promote hair growth.

Hair grows from follicles in the skin that extend from the epidermis (the outmost layer of skin) to the dermis (the deepest layer of the skin). Hair length and thickness mainly depends on two biological processes: hair follicle regeneration and the synthesis of hair fibers.

The hair cycle is a stem cell-mediated process that occurs in adult skin and involves the periodic destruction and regeneration of hair follicles. It consists of three defined stages: growth (anagen), followed by regression (catagen) and rest (telogen). Anagen phase is the longest stage in the hair cycle. At this stage the cell proliferation begins, the follicle is created, and the hair is formed consecutively. In the catagen phase, there is a pause of proliferation and an increase in cellular apoptosis. The hair stops growing, and the follicle shortens. Finally, in the telogen phase, described as a resting phase, the hair breaks down. [Alonso L and Fuchs E (2006) The hair cycle. J. Cell Sci. 119:391-393.]

Growth of a new hair requires reentry into the anagen phase, a process involving the activation of multipotent epithelial stem cells residing in a specialized part of the follicle outer root sheath (ORS) known as the bulge. Dermal cells known as Hair Follicle Derma Papilla Cells (HFDPC) reside in the bulge [Beaudoin G M 3rd, Sisk J M, Coulombe P A, and Thompson C C. (2005) Hairless triggers reactivation of hair growth by promoting Wnt signaling. Proc Natl Acad Sci USA. 102:14653-14658]. HFDPC in the adult hair follicle play a crucial role in the hair growth cycle by inducing follicle development [Driskell R R, Clavel C, Rendl M and Watt F M. (2011) Hair follicle dermal papilla cells at a glance. J. Cell Sci. 124:1179-1182].

Hair fiber is composed mostly of proteins (65-95% of hair weight), with keratin being the most abundant. This fiber consists of three general layers: cortex, cuticle, and medulla. The central medulla contains polygonal cells with a sponge-like appearance. Around the medulla, the cortex forms a layer of cornified fibrous cells with a longitudinal orientation and are packed with keratin filaments. In addition, the cortex contains melanosomes which determine the color of the hair fiber. The outermost layer of the hair fiber, the cuticle, consists of multiple layers of corneocytes. It is thin and translucent allowing light to penetrate to the cortex pigments. The keratin proteins which form this complex structure of hair fiber are the result of proliferative activity of keratinocytes situated in the hair follicle. These keratinocytes are found in the dermal papilla (DP) and together with other cells involved in this proliferative activity develop the hair growth.

Compositions and methods for enhancing the growth of hair are known in the art. However, known methods can be ineffective or can have undesirable side effects.

Drugs, including Minoxidil® (Rogaine), Finasteride® (Propecia) and Dutasteride® (Avodart) are approved treatments for hair loss. However, they efficacy is mainly limited to hormonal-related alopecia (i.e., androgenic alopecia) and they require medical prescription. There is also a growing concern about the use of synthetic chemicals in cosmetic preparations applied to the human body. Developing cosmetic preparations for areas in close proximity to the human eye, such as, for example, preparations designed for promoting the growth of eyelash or eyebrow hairs, presents particular challenges because the human eyeballs and surrounding membranes are particularly sensitive as compared to the human skin on other areas of the body. Although cosmetics for eyelashes are not intended to be applied directly to the eyeball or eye membranes, a user may accidentally transfer some of the cosmetic preparation into the eyeball during application. Moreover, there is a growing concern about the environmental impacts caused by the production of synthetic chemical products utilized in cosmetic products, for example, by washing cosmetic products off the body after use or by disposing unused amounts of such products in landfills.

Volumizing mascara for eyelashes is one of the most common products for enhancing the appearance of eyelashes. However, those with short eyelashes have trouble in achieving bold-looking eyelashes by mascara alone and may choose to use extensions. Eyelash extensions often involve the use of harsh adhesives to bind the extensions to the eyelid near the eyelash line, and thus can cause damage to natural eyelashes. Different risks associated with the use of eyelash extensions have been reported such as swelling of the eyelid, loss of eyelashes, skin irritation of the eyelid or infection of the eyelid. There is a desire for a mascara composition that can enhance the appearance of eyelashes that promotes the growth of eyelashes.

Micropigmentation and microblading are tattoo procedures used to improve the density and definition of eyebrows. However, they attract the inconveniences associated with any tattoo procedure, such as pain and risk of infection.

Natural, sustainable, and efficacious solutions to the above-identified problems are needed. Some herbal preparations have been proposed to promote hair growth in eyelashes and eyebrows; however, their effectiveness can be limited.

EP2764894B1 provides extracts from the leaf of the blackberry plant for inducing hair growth. The extract is said to induce a visible telogen phase (i.e., shedding of club hairs) prior to rapid entry into the anagen phase (i.e., active growth phase of hair follicles). Extractants used for obtaining the extract include ethanol or a combination of ethanol and alcohol. Ethanol and alcohol extractions have inherent flammability hazards in manufacturing and production as well as transportation logistics. Ethanol is considered a green solvent and has good extraction capabilities but is undesired by some segments of the cosmetics market and for some consumers.

US20190224160A1 discloses compositions for facilitating eyelash growth comprising at least forskolin derived exclusively from Coleus forskohlii in combination with a keratinocyte growth stimulator. The keratinocyte growth stimulator comprises at least one plant extract selected from a group comprising: Tussilago farfara flower extract, Achillea millefolium extract, Cinchona succirubra bark extract, Nasturtium officinale, Tropaeolum majus.

WO2017032711 discloses a cosmetic composition for promoting the growth of eyelashes, the composition comprising a botanical extract of Vigna radiata, an extract of the sprout of the mung bean, and a peptide comprising the amino acid sequence Gly-His-Lys from N-terminal to C-terminal end.

Myrothamnus sp. is a drought tolerant bush native to South Africa belonging to a family of plants know as ‘resurrection plants’. This botanical has adapted to the seasonal wet and dry periods of the region. In the dry season, the leaves dry up as the plant becomes dormant while it waits for the rains to return. At the first rainfall, the plant is resurrected and springs to life, quickly hydrating its leaves and flowering. Resurrection plants have a number of ways of avoiding mechanical and drought stress, and have been of interest to the scientific community looking for active substances with cosmetic and/or pharmaceutical properties. Myrothamnus is consumed as a tea in southern Africa and essential oils are extracted for use as well. Its use in local food and medicine traditions is well known in South Africa. Myrothamnus has been used in the cosmetic industry for skin care and is the subject of several patents/patent applications, such as US200701341934A1, FR29978536B1 and KR1305698B1.

There is a need to find natural, cosmetic, non-therapeutic treatments that can increase hair growth and thickness, particularly in relation to hair in sensitive facial areas such as close to the eyes, i.e., hair of the eyelashes and/or eyebrows. Desirably, such treatments would have little or no negative side effects, and/or would provide beautifying benefits for the hair and its associated skin.

The present invention sets out to meet some or all of the above-identified needs and to solve some or all of the above-identified problems.

### SUMMARY OF THE INVENTION

In a first aspect, the invention provides for the use of an extract of Myrothamnus sp. for promoting hair growth, preventing hair loss, and/or increasing the thickness of hair. In particular, the use can be a cosmetic, non-therapeutic use. In particular, the hair can be hair of the eyelashes and/or of the eyebrows. The invention provides for the cosmetic, non-therapeutic use of an extract of Myrothamnus sp. for increasing the length of eyelashes and/or increasing the thickness (i.e., increasing the density of hairs) of the eyebrows. The use of a Myrothamnus extract is especially advantageous, since consumers have a special interest in cosmetic treatments considered as “natural” as generally such treatments are more environmentally friendly.

In another aspect, the invention provides for a method of promoting and/or increasing hair growth, preventing hair loss, and/or increasing the thickness of hair, comprising administering a botanical extract of Myrothamnus sp. to a subject. In particular, the method can be a cosmetic, non-therapeutic method. In particular, the hair can be the hair of the eyelashes or of the eyebrows. The invention provides for a cosmetic, non-therapeutic method increasing the length of eyelashes and/or increasing the thickness (i.e., increasing the density of hairs) of the eyebrows comprising administering a botanical extract of Myrothamnus sp. to a subject.

In another aspect, the invention provides a process for obtaining a botanical extract from Myrothamnus sp. comprising subjecting plant material of Myrothamnus sp. to an extraction with subcritical water, wherein the extraction comprises the steps of:
i) contacting the plant material with subcritical water at a temperature of from at least about 120° C. and a pressure suitable to maintain the water in a liquid state for a period of at least 10 minutes to form an aqueous botanical extract; and
ii) separating the plant material from the aqueous botanical extract.

In another aspect, the invention provides for an aqueous botanical extract obtained/obtainable by the afore-mentioned process for obtaining a botanical extract from Myrothamnus sp.

In another aspect, the invention provides for a cosmetic composition comprising the aqueous botanical extract and at least one cosmetically acceptable excipient or ingredient.

In another aspect, the invention provides for a mascara composition comprising a botanical extract from Myrothamnus sp. and at least one pigment.

### DETAILED DESCRIPTION OF THE INVENTION

The invention is based on the finding of surprising properties of a botanical extract from plants of genus Myrothamnus i.e., Myrothamnus sp., which properties lend the extract to cosmetic, non-therapeutic applications.

### Definitions

In the context of this invention “hair” includes the hair of the scalp, the skin, the eyelashes, the eyebrows, moustache region and/or beard region of a subject. In particular, hair is the hair of the eyebrows and/or the eyelashes. “Skin” is understood to be the layers which comprise it, from the uppermost layer or stratum corneum to the lowermost layer or hypodermis, both inclusive. These layers are composed of different types of cells such as keratinocytes, fibroblasts, melanocytes, mast cells, neurons and/or adipocytes among others. The term “skin” includes the skin of mammals, for example, the skin of humans, and includes skin comprising hair.

The term “about” as used herein, e.g., when referring to a measurable value (such as an amount or weight of a particular component or temperature), refers to variations of ±20%, ±10%, ±5%, ±1%, ±0.5%, or, particularly, ±0.1% of the specified amount.

As used herein, the term “comprising”, which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

### The Extract

The present invention relates to the use a botanical extract of Myrothamnus sp. The terms “botanical extract” and “extract” are used interchangeably herein and refer to a product containing one or more compounds that have been extracted from plant material of Myrothamnus sp. Specifically, the product is obtained by subjecting plant material of Myrothamnus sp. to a solid/liquid extraction whereby one or more phytochemical compounds contained in the plant material (the solid) are extracted from the plant material into the solvent (the liquid); they are dissolved out of the plant material into the solvent. The product can be a composition comprising the extraction solvent and the one or more phytochemical compounds. The solid/liquid extraction process can involve a separation step, optionally, followed by one or more purification steps. Thus, the extract can be the product obtained after the separation step or the product obtained after the purification step. In the separation step, the composition comprising the solvent and the one or more phytochemical compounds is separated from the plant material. The extract is the separated-out composition comprising the extraction solvent and the one or more phytochemical compounds. The solubility of the phytochemical compounds in the solvent may be temperature dependent and therefore this composition may be a solution, or if it cools to a temperature lower than that used for extraction, some of the phytochemical compounds may precipitate out. When the solvent is water, the composition comprising the extraction solvent and the one or more phytochemical compounds is also referred herein as the “aqueous botanical extract” or the “aqueous extract”. Purification of the composition comprising the solvent and the one or more phytochemical compounds (for example by fractionation, concentration, or drying) provides an extract that can be, for example, a concentrate (e.g., in a viscid form) or a solid (e.g., a powder).

Myrothamnus sp. is a genus of flowering plants of small xerophytic shrubs, including two species, namely M. flabellifolia and M. moschata. Particularly, the extract can be an extract of M. flabellifolia. M. flabellifolia is also referred in the literature as M. flabellifolius.

Typically, the plant material from Myrothamnus sp. includes aerial parts of the plant, particularly the stems and/or the leaves. The plant material can comprise stems and/or leaves as a major component, e.g., stems and/or leaves constitute more than 50, 80 or 90% by weight of the plant material. The plant material can comprise stems and/or leaves only, i.e., these are the only components, no other plant material is present.

Typically, the plant material is in dried form. The plant material can be dried so that it is easier to homogenize, manipulate and store. By “dried plant material” is meant plant material that has a water content of less than 10% of water by weight, less than 5% of water by weight, less than 2% of water by weight, or less than 1% of water by weight. The water content can be measured by AOAC (Association of Official Analytical Chemists) Official Method AOAC 934.06-1934 (1996), for example. However, plant material that has not been dried may also be used.

Advantageously, plant material is ground or crushed before extraction. Ground plant material can have, for example, a particle size ranging from 100 pm to 50 mm, with an average particle size of from 0.01 or 0.1 to 10 mm. More particularly, the average particle size can range from 0.2 to 5 mm. The average particle size can be determined by conventional methods such as those involving sieve analysis, for example. Any suitable grinding/crushing technique known in the art may be used to obtain the desired particle size of the plant material. A particularly suitable plant material is dried plant material in powder form having, for example, particle sizes as mentioned above.

The plant material can be mixed with a neutral material, to make it drier and/or more porous for solvent extraction. Suitable neutral materials include graphene, silica gel, C18-resins, diatomaceous earth and neutral alumina.

Suitable solvents for use in the extraction include water, lower alcohols of 1 to 4 carbon atoms (e.g., methanol, ethanol, butanol, etc.) and glycols and combination thereof. Advantageously, the extract from Myrothamnus sp. can be obtained using water as the only extraction solvent. In this case, compounds in the plant material dissolve out of the plant material into the water to form an aqueous extract (also referred to herein in as an aqueous botanical extract). The aqueous extract can be purified to form an extract which is a concentrate or a powder, for example. Thus, advantageously, the extract from Myrothamnus sp. can be obtained avoiding the use of organic solvents during the extraction process.

Any conventional solid/liquid extraction method may be used to obtain the botanical extract from the plant material of Myrothamnus sp., for example Soxhlet, percolation, and maceration. The temperature of the solvent should be chosen as appropriate for that solvent. If water is used as the extraction solvent, preferably it is used at a temperature of greater than 60° C. Typically, the ratio of dried plant material/solvent (weight of dried plant material in g/volume of solvent in mL) is in a range of from 1:5 to 1:50, or from 1:10 to 1:30 or from 1:15 to 1:25 or from 1:5 to 1:15. Particularly, the ratio is 1:10 or 1:20.

In one embodiment, the extract of Myrothamnus sp. is obtained by subjecting plant material of Myrothamnus sp. to a solid/liquid extraction using subcritical water as the extraction solvent. Subcritical water is water that is held in the liquid state by pressure at a temperature higher than its natural boiling point of 100° C. (i.e., higher than its boiling point at atmospheric pressure). Subcritical water can have a temperature up to its critical point temperature of 374° C. Subcritical water is also referred to as “pressurized low polarity water”, “pressurized hot water” or “compressed hot water”. Heating water under pressure to temperatures above its boiling point results in the alteration of its key properties such as polarity. Preferably, when subcritical water is used as the extraction solvent, it is the only extraction solvent used in the process, i.e., the extraction is performed with subcritical water in the absence of any other solvent (organic or inorganic).

More specifically, the extract from Myrothamnus sp. can be obtained by subjecting the plant material of Myrothamnus sp. to an extraction with subcritical water, wherein the extraction comprises a step of contacting the plant material with subcritical water at a temperature of at least about 120° C. and a pressure suitable to maintain the water in a liquid state for a period of at least 10 min so as to form an aqueous botanical extract. More specifically, the extract from Myrothamnus sp. can be obtained by a process comprising the steps of:
i) contacting a plant material of Myrothamnus sp. with subcritical water at a temperature of at least 120° C. and a temperature pressure suitable to maintain the water in a liquid state for a period of at least 10 min to form an aqueous botanical extract; and
ii) separating the plant material from the aqueous botanical extract.

Typically, the ratio of dried plant material/subcritical water ratio (weight of dried plant material in g/volume of water in mL) is in a range of from 1:5 to 1:50, or from 1:10 to 1:30 or from 1:15 to 1:25 or from 1:5 to 1:15. Particularly, the ratio can be 1:10 or 1:20.

Particularly, the extraction can be performed using subcritical water at a temperature from about 120 to about 220° C., from about 130 to about 180° C., from about 140 to about 160° C., or from about 145 to about 155° C. Particularly, the subcritical water can be at a temperature of about 150° C.

When subcritical water is the extraction solvent, the extraction is carried out in a pressurizable container, typically a stainless steel container. During the extraction, the pressure in the pressurizable container must be such that the subcritical water is maintained in a liquid state. The pressure required to achieve this will vary depending on the temperature of the water. The skilled person would be able to determine the pressure required. Typically, the pressure will range from 0.5 MPa to 20 MPa. The pressure can be at least about 1 MPa. The pressure can be from about 5 to about 15 MPa or from about 8 to about 13 MPa. The pressure can be 10, 11 and 12 MPa.

The step of contacting the plant material with subcritical water is carried out for a period of at least about 10 minutes. This period of time is the time that the plant material is in contact with subcritical water, whether the water is static or flowing over plant. This period is also referred to herein as the extraction time. The extraction time will vary, for example, depending on the amount of plant material and water used. Typically, the extraction time will vary from 10 minutes to about 5 hours. For example, the extraction time can be at least about 15 minutes, or at least about 30 minutes, or at least about 1 hour or at least about 2 hours, or at least about 3 hours. In particular, the extraction time is from about 10 minutes to about 2 hours, more particularly 20 minutes to 90 minutes. The extraction time can be from 45 to 75 minutes or to about 1 hour.

The subcritical water extraction can be performed in batch mode (also referred as ‘static mode’) or in dynamic mode (also referred as ‘flow-though’). In batch mode, the plant material is exposed to (i.e., contacted with) the subcritical water in batches. In batch mode, the volume of subcritical water used is the total volume of subcritical water used for all the batches for the sample of plant material. In batch mode, the extraction time is the total time that the plant material is exposed to the subcritical water over all the batches for the sample of plant material. In dynamic mode, the plant material is exposed to a continuous flow of water. In dynamic mode, the volume of subcritical water used is the total volume of subcritical water used for the sample of plant material. In dynamic mode, the extraction time is the total time that the plant material is exposed to the flow of subcritical water for the sample of plant material. For faster, larger scale production of the botanical extract, advantageously, the extraction is performed in dynamic mode. The extraction may be conducted in any system known in the art allowing subcritical water extraction, and including a system comprising a batch extractor or a continuous extractor.

During the step of contacting the plant material with subcritical water, compounds are extracted from the plant material into the subcritical water. More specifically, components of the plant material dissolve in the subcritical water thus forming an aqueous botanical extract. Similarly, conventional solvent extraction methods using water as the solvent will form an aqueous botanical extract. The solvent extraction process can comprise a step of separating the plant material (i.e., any undissolved plant material) from the botanical extract. Typically, this step is straightforward. For example, in the case of subcritical water extraction, it can simply involve releasing the aqueous botanical extract from the pressurizable container while retaining the plant material in the pressurizable container. Other suitable separation techniques are known in the art and include, for example, filtration, sedimentation, decantation or centrifugation. Filtration can be carried out using filters having a pore size of lower than 1000 μm, or lower than 20 μm, or lower than 10 μm, or lower than 1 μm, or lower than 0.1 μm. Filtration may be performed in successive filtration operations, for instance using filters of decreasing pore size. The residue (undissolved plant material) left after filtration may be re-contacted with more extraction solvent. This filtration-recontacting step may be carried out once or may be repeated, for example, repeated 1 to 5 times. In the case of subcritical water extraction, preferably, separation is carried out before the aqueous botanical extract cools and precipitates extract components.

After the botanical extract has been separated from the plant material, it can be purified. In the context of this invention, “purifying” means purification, partial purification, and/or fractionation. There are a large number of techniques well known in the art for the purification of botanical extracts. Some non-limiting examples include solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilization, evaporation, precipitation with various “carriers” (including PVPP, carbon, antibodies, etc.), or various combinations thereof. In one embodiment, the botanical extract is concentrated so as to form a concentrate of the botanical extract. Alternatively, the botanical extract is dried so as to form a solid form of the botanical extract. The botanical extract may be dried so as to contain no more than 10% of water by weight, no more than 5% of water by weight, no more than 2% of water by weight or no more than 1% of water by weight. This can be measured by moisture content determination AOAC (2000), for example. Suitable concentration and/or drying methods are well known in the art. Examples include, but are not limited to, reduced pressure evaporation, evaporation, reduced pressure distillation, distillation, oven drying, sun drying, and lyophilization (i.e., freeze-drying), spray drying, atomization or fluidized bed dryer. Concentration or drying can be performed on the botanical extract with or without a carrier or other excipients. The extract can be a concentrate, or a solid, for example an amorphous solid, a crystalline or part-crystalline solid, optionally in the form of a powder.

Thus, the invention also provides a botanical extract of Myrothamnus sp. obtained/obtainable by any of the processes described above. In particular, the botanical extract of Myrothamnus sp. is obtained/obtainable in any of the processes described above which involve extraction with water. The botanical extract of Myrothamnus sp. may be obtained/obtainable in any of the processes described above which involve extraction with subcritical water. Details of the extraction processes as described herein can be applied or incorporated into the definition of the botanical extract provided herein.

The botanical extract comprises phytochemicals which are chemical compounds that are produced by plants and can include polyphenols, amino acids, organic acids, and sugars for example.

The botanical extract may comprise: miquelianin and kaempferol-3-O-glucuronide; or miquelianin, kaempferol-3-O-glucuronide and trehalose; or miquelianin, kaempferol-3-O-glucuronide, trehalose and luteolin-7-O-glucuronide; or miquelianin, kaempferol-3-O-glucuronide, trehalose, luteolin-7-O-glucuronide and arbutin; or miquelianin, kaempferol-3-O-glucuronide, trehalose, luteolin-7-O-glucuronide, arbutin and kaempferol-3-O-glucoside; or miquelianin, kaempferol-3-O-glucuronide, trehalose, luteolin-7-O-glucuronide, arbutin, kaempferol-3-O-glucoside and quercetin-3-O-galactoside; or miquelianin, kaempferol-3-O-glucuronide, trehalose, luteolin-7-O-glucuronide, arbutin, kaempferol-3-O-glucoside, quercetin-3-O-galactoside and syringic acid; or miquelianin, kaempferol-3-O-glucuronide, trehalose, luteolin-7-O-glucuronide, arbutin, kaempferol-3-O-glucoside, quercetin-3-O-galactoside, syringic acid and quercitrin; or miquelianin, kaempferol-3-O-glucuronide, trehalose, luteolin-7-O-glucuronide, arbutin, kaempferol-3-O-glucoside, quercetin-3-O-galactoside, syringic acid, quercitrin, and isoquercitrin; or miquelianin, kaempferol-3-O-glucuronide, trehalose, luteolin-7-O-glucuronide, arbutin, kaempferol-3-O-glucoside, quercetin-3-O-galactoside, syringic acid, quercitrin, isoquercitrin and tryptamine.

The botanical extract may be obtained/obtainable by solid/liquid extraction using water as a solvent and may comprise: miquelianin and kaempferol-3-O-glucuronide; or miquelianin, kaempferol-3-O-glucuronide and trehalose; or miquelianin, kaempferol-3-O-glucuronide, trehalose and luteolin-7-O-glucuronide; or miquelianin, kaempferol-3-O-glucuronide, trehalose, luteolin-7-O-glucuronide and arbutin; or miquelianin, kaempferol-3-O-glucuronide, trehalose, luteolin-7-O-glucuronide, arbutin and kaempferol-3-O-glucoside; or miquelianin, kaempferol-3-O-glucuronide, trehalose, luteolin-7-O-glucuronide, arbutin, kaempferol-3-O-glucoside and quercetin-3-O-galactoside; or miquelianin, kaempferol-3-O-glucuronide, trehalose, luteolin-7-O-glucuronide, arbutin, kaempferol-3-O-glucoside, quercetin-3-O-galactoside and syringic acid; or miquelianin, kaempferol-3-O-glucuronide, trehalose, luteolin-7-O-glucuronide, arbutin, kaempferol-3-O-glucoside, quercetin-3-O-galactoside, syringic acid and quercitrin; or miquelianin, kaempferol-3-O-glucuronide, trehalose, luteolin-7-O-glucuronide, arbutin, kaempferol-3-O-glucoside, quercetin-3-O-galactoside, syringic acid, quercitrin, and isoquercitrin; or miquelianin, kaempferol-3-O-glucuronide, trehalose, luteolin-7-O-glucuronide, arbutin, kaempferol-3-O-glucoside, quercetin-3-O-galactoside, syringic acid, quercitrin, isoquercitrin and tryptamine.

The botanical extract may comprise: miquelianin, kaempferol-3-O-glucuronide, trehalose, luteolin-7-O-glucuronide and isorhamnetin-3-O-glucoside; or miquelianin, kaempferol-3-O-glucuronide, trehalose and luteolin-7-O-glucuronide, isorhamnetin-3-O-glucoside and naringenin; or miquelianin, kaempferol-3-O-glucuronide, trehalose, luteolin-7-O-glucuronide, isorhamnetin-3-O-glucoside, naringenin and piceid; or miquelianin, kaempferol-3-O-glucuronide, trehalose, luteolin-7-O-glucuronide, isorhamnetin-3-O-glucoside, naringenin, piceid and coniferaldehyde; or miquelianin, kaempferol-3-O-glucuronide, trehalose, luteolin-7-O-glucuronide, isorhamnetin-3-O-glucoside, naringenin, piceid, coniferaldehyde and naringenin-7-O-glucoside.

The botanical extract may be obtained/obtainable by solid/liquid extraction using water as a solvent and may comprise: miquelianin, kaempferol-3-O-glucuronide, trehalose, luteolin-7-O-glucuronide and isorhamnetin-3-O-glucoside; or miquelianin, kaempferol-3-O-glucuronide, trehalose and luteolin-7-O-glucuronide, isorhamnetin-3-O-glucoside and naringenin; or miquelianin, kaempferol-3-O-glucuronide, trehalose, luteolin-7-O-glucuronide, isorhamnetin-3-O-glucoside, naringenin and piceid; or miquelianin, kaempferol-3-O-glucuronide, trehalose, luteolin-7-O-glucuronide, isorhamnetin-3-O-glucoside, naringenin, piceid and coniferaldehyde; or miquelianin, kaempferol-3-O-glucuronide, trehalose, luteolin-7-O-glucuronide, isorhamnetin-3-O-glucoside, naringenin, piceid, coniferaldehyde and naringenin-7-O-glucoside.

The botanical extract may comprise: miquelianin, kaempferol-3-O-glucuronide, trehalose, luteolin-7-O-glucuronide, arbutin and isorhamnetin-3-O-glucoside; or miquelianin, kaempferol-3-O-glucuronide, trehalose and luteolin-7-O-glucuronide, arbutin, isorhamnetin-3-O-glucoside and naringenin; or miquelianin, kaempferol-3-O-glucuronide, trehalose, luteolin-7-O-glucuronide, arbutin, isorhamnetin-3-O-glucoside, naringenin and piceid; or miquelianin, kaempferol-3-O-glucuronide, trehalose and luteolin-7-O-glucuronide, arbutin, isorhamnetin-3-O-glucoside, naringenin, piceid and coniferaldehyde; miquelianin, kaempferol-3-O-glucuronide, trehalose, luteolin-7-O-glucuronide, arbutin, isorhamnetin-3-O-glucoside, naringenin, piceid, coniferaldehyde and naringenin-7-O-glucoside.

The botanical extract may be obtained/obtainable by solid/liquid extraction using water as a solvent and may comprise: miquelianin, kaempferol-3-O-glucuronide, trehalose, luteolin-7-O-glucuronide, arbutin and isorhamnetin-3-O-glucoside; or miquelianin, kaempferol-3-O-glucuronide, trehalose and luteolin-7-O-glucuronide, arbutin, isorhamnetin-3-O-glucoside and naringenin; or miquelianin, kaempferol-3-O-glucuronide, trehalose, luteolin-7-O-glucuronide, arbutin, isorhamnetin-3-O-glucoside, naringenin and piceid; or miquelianin, kaempferol-3-O-glucuronide, trehalose and luteolin-7-O-glucuronide, arbutin, isorhamnetin-3-O-glucoside, naringenin, piceid and coniferaldehyde; miquelianin, kaempferol-3-O-glucuronide, trehalose, luteolin-7-O-glucuronide, arbutin, isorhamnetin-3-O-glucoside, naringenin, piceid, coniferaldehyde and naringenin-7-O-glucoside.

### Compositions

As described above, the botanical extract obtained from Myrothamnus sp. by extraction with water can be in aqueous form, i.e., an aqueous botanical extract. The extracted components from the plant material may or may not be fully dissolved in the aqueous botanical extract. Advantageously, the aqueous botanical extract can be mixed with a water-miscible organic solvent so as to keep the extracted components in solution. The process for obtaining the botanical extract of the invention can include a step comprising mixing an aqueous botanical extract as defined herein with a water-miscible organic solvent. The resultant composition comprises an aqueous botanical extract and a water-miscible organic solvent. Water can be added to this composition to obtain the required concentration of water-miscible solvent, for example. The processes for obtaining the botanical extract of Myrothamnus sp. described herein can include a step comprising mixing a botanical extract as defined herein with a water-miscible organic solvent and, optionally, water. The resultant composition comprises the botanical extract with a water-miscible organic solvent and, optionally, water. These solutions can be used as “stock solutions” and can be used in the preparation of cosmetic compositions, for example.

The water-miscible organic solvent is preferably a cosmetically acceptable organic solvent and can be a polyol. Suitable polyols included glycols which are organic compounds containing two alcohol functions (—OH groups), such as: C2-C10 aliphatic hydrocarbyl diols or the triol, glycerin. C2-C10 aliphatic hydrocarbyl diols include C2-C10 or C2-C8 alkanediols in each isomeric form and which can be substituted or unsubstituted. When substituted, the alkane diols can be substituted with one or more substituents independently selected from halo, hydroxyl, ester, nitro, cyano, haloalkyl, sulfonyl and carbonyl groups, for example. The water-miscible organic solvent can be chosen from: 1,2-propanediol, ethylene glycol, diethylene glycol, propylene glycol, dipropylene glycol, butylene glycol, pentylene glycol, glycerol or caprylyl glycol and mixtures thereof. Particularly, the polyol is glycerol (also referred to herein as glycerine or glycerin).

The composition comprising the botanical extract and the polyol, and optional (additional) water, can have a concentration of polyol of at least 50%, or from 50% to 90%, or from 50 to 80%, or from 55 to 80% by weight based on the total weight of the composition. Typically, the balance is made up with the botanical extract and water. For example, the composition can comprise up to 50% water by weight. Preferably, the polyol is glycerin. The composition comprising the botanical extract and the polyol and water can have a concentration of polyol is at least 50%, or from 50% to 90%, or from 50 to 80%, or from 55 to 80% by weight based on the total weight of the composition. Typically, the balance is made up with the botanical extract and water. For example, the composition can comprise up to 50% water by weight. Preferably, the polyol is glycerin.

The extract or a composition comprising the extract, a water-miscible organic solvent and, optionally, water, as described above, can be incorporated into compositions suitable for administration to a subject.

The composition may be a cosmetic composition together comprising the extract or a composition comprising the extract, a water-miscible organic solvent and, optionally, water, as described above, along with at least one cosmetically acceptable excipient or adjuvant. These compositions can be prepared by conventional means known to persons skilled in the art [“Harry's Cosmeticology”, Seventh edition, (1982), Wilkinson J. B., Moore R. J., ed. Longman House, Essex, GB].

The cosmetic composition contains a cosmetically effective amount of the botanical extract which should be administered, as well as their dosage, will depend on numerous factors, including age, state of the patient, the nature or severity of the condition, disorder or disease to be treated and/or cared for, the route and frequency of administration and of the particular nature of the compounds to be used.

The terms “cosmetically effective amount” is understood to mean a non-toxic but sufficient amount of extract of the invention to provide the desired effect. The extracts or stock solutions of the invention are used in cosmetic compositions of this invention at cosmetically effective concentrations to achieve the desired effect; for example in amounts with respect to the total weight of the composition of: from 0.00000001% (in weight) to 20% (in weight); from 0.000001% (in weight) to 15% (in weight), from 0.00001% (in weight) to 10% (in weight); or from 0.0001% (in weight) to 5% (in weight); or from 0.1 to 4% (in weight); or from 1 to 3% (In weight).

In one embodiment, the extract or the composition comprising the extract, a water-miscible organic solvent and, optionally, water, as described above, is present in an amount of 2% (by weight) in the cosmetic composition.

The cosmetic compositions of the invention can be compositions for topical application which optionally include cosmetically or pharmaceutically acceptable excipients necessary for formulating the desired administration form. Topical compositions are compositions suitable for the topical application to mammalian keratinous tissue such as skin having hair, particularly to the human scalp. In particular, the topical compositions are hair care compositions such as conditioners, treatments, hair tonics, styling gels, mousses, shampoos, hair sprays, pomades, setting lotions, coloring and permanent waving compositions. Of particular interest for the purpose of the present invention are tonics, conditioners, treatments, and styling gels which may be in the form of a gel, a lotion, a tincture, a spray, a mousse, a cleansing composition or a foam and which may be applied according to individual needs, e.g., once daily as a lotion, tincture, mousse or spray; or once or twice weekly as a conditioner or treatment. Particularly suitable compositions in the context of this invention are lotions, a shampoo, a serum or eyelashes mascara. Preferably, the cosmetic composition is a serum or an eyelash mascara. The term “serum” is well known by the person skilled in the art and refers to compositions that are clear, gel-based or liquid. Serum has a fluid texture and is more concentrated in active agents than a standard care product. The term “mascara” as used herein refers to cosmetic products used to enhance the eyelashes. It may darken, thicken, lengthen, and/or define the eyelashes, for example.

The cosmetic composition can be a mascara composition Mascara compositions of the present invention are similar to presently known mascaras in that they incorporate the basic formulation elements of a mascara.

The mascara composition comprises (i) the botanical extract as described herein or a composition comprising the botanical extract, a water-miscible organic solvent and, optionally, water, as described herein; and (ii) at least one cosmetically acceptable excipient or adjuvant. The at least one cosmetically acceptable excipient or adjuvant comprises at least one wax and, optionally, at least one pigment. The mascara composition comprises (i) the botanical extract as described herein or a composition comprising the botanical extract, a water-miscible organic solvent and, optionally, water, as described herein; and at least one wax and, optionally, at least one pigment. Typically, the mascara composition is an emulsion and comprises a liquid phase (e.g., water) and at least one emulsifier. The mascara composition can further comprise at least one rheology modifier, such as a thickener and/or a film forming agent.

As used herein the term “wax” is intended to mean a lipophilic fatty compound that is solid at room temperature (about 25° C.) and atmospheric pressure (760 mmHg, i.e., 105 Pa), which undergoes a reversible solid/liquid change of state and which has a melting point of greater than 30° C., and in some embodiments, greater than about 55° C., up to about 120° C., or even as high as about 200° C. The term wax includes waxes of animal origin, waxes of plant origin, waxes of mineral origin and waxes of synthetic origin. Examples of waxes of animal origin include beeswaxes and lanolin waxes. Examples of waxes of plant origin include rice waxes, carnauba wax, candelilla wax, ouricury wax, cork fiber waxes, sugar cane waxes, Japan waxes, sumac wax and cotton wax, sunflower wax. Examples of waxes of mineral origin include paraffins, microcrystalline waxes, montan waxes, ozokerites and ceresin. Examples of waxes of synthetic origin include polyolefin waxes, e.g., polyethylene waxes, waxes obtained by Fischer-Tropsch synthesis, waxy copolymers and their esters, and silicone and fluoro waxes. Suitable waxes include Oryza sativa Bran Cera, Copernicia cerifera wax and Shellac wax. The term wax may further include high melting point hydrogenated oils of animal or plant origin. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils. The mascara composition can comprise at least one wax, wherein the total amount of wax in the composition is from 0.5 to 20 wt %, or 1 to 10 wt %, or from 2 to 8 wt %, or from 4 to 7 wt %, based on the total weight of the mascara composition.

The pigment may be mineral and/or organic, and coated or uncoated. Among the mineral pigments, mention may be made of metal oxides, in particular titanium dioxide, optionally surface-treated, zirconium, zinc or cerium oxide, and also iron oxide, particularly black iron oxide, titanium oxide or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum. The mascara composition can comprise at least one pigment, wherein the total amount of pigment in the composition is from 1 to 20 wt %, or 5 to 15 wt %, or from 8 to 12 wt %, based on the total weight of the mascara composition.

Suitable emulsifiers for use in the mascara composition include anionic, amphoteric, and non-ionic emulsifiers suitable for emulsifying fatty compounds in a water phase. In certain embodiments of the invention, the emulsifiers are selected from fatty acids, fatty acid esters of glycerol and/or polyalkylene glycols, amphoacetates, and alkyl phosphates. In certain embodiments of the invention, the emulsifiers are selected from stearic acid, glyceryl stearate, peg-200 glyceryl stearate, steareth-2, steareth-20 isoceteth-20, and potassium cetyl phosphate, and disodium cocoamphoacetate. Suitable emulsifiers include Glucate™ SS, Arlatone™ MAP 160, and a mixture thereof. The mascara composition can comprise at least one emulsifier, wherein the total amount of emulsifier in the composition is from 0.01 to 8 wt %, or from 0.05 to 8 wt %, or from 0.05 to 5 wt %, 0.05 to 0.5 wt %, or from 0.1 to 0.20 wt %, based on the total weight of the mascara composition.

The mascara composition may comprise a rheology modifier. Rheology modifiers include viscosity-increasing polymeric natural and derivatized gums, resin thickeners, gellants or suspending agents. To increase the viscosity, the composition may include one or more rheology modifiers, which can be synthetic or natural.

Examples include fatty alcohols, such as C10-C32 alcohols, e.g., C12-C22 alcohols, natural oils, and polymers of acrylic acid and/or methacrylic acid, such as carbomers. Exemplary natural oils include mineral oils (mainly C15-C40 linear and branched aliphatic alkanes, with minor amounts of cycloalkanes), natural derived esters, natural alkanes, or vegetable oils. Exemplary synthetic rheology modifiers include acrylic based polymers and copolymers. One class of acrylic based rheology modifiers are the carboxyl functional alkali-swellable and alkali-soluble thickeners (ASTs) produced by the free-radical polymerization of acrylic acid alone or in combination with other ethylenically unsaturated monomers. The polymers can be synthesized by solvent/precipitation as well as emulsion polymerization techniques. Exemplary synthetic rheology modifiers of this class include homopolymers of acrylic acid or methacrylic acid and copolymers polymerized from one or more monomers of acrylic acid, substituted acrylic acid, and salts and C1-C30 alkyl esters of acrylic acid and substituted acrylic acid. As defined herein, the substituted acrylic acid contains a substituent positioned on the alpha and/or beta carbon atom of the molecule, wherein in one aspect the substituent is independently selected from C1-4 alkyl, —CN, and —COOH. Optionally, other ethylenically unsaturated monomers such as, for example, styrene, vinyl acetate, ethylene, butadiene, acrylonitrile, as well as mixtures thereof can be copolymerized into the backbone. The foregoing polymers are optionally crosslinked by a monomer that contains two or more moieties that contain ethylenic unsaturation. In one aspect, the crosslinker is selected from a polyalkenyl polyether of a polyhydric alcohol containing at least two alkenyl ether groups per molecule. Other Exemplary crosslinkers are selected from allyl ethers of sucrose and allyl ethers of pentaerythritol, and mixtures thereof. These polymers are more fully described in U.S. Pat. Nos. 5,087,445; 4,509,949; and 2,798,053.

In one aspect, the AST rheology modifier or thickener is a crosslinked homopolymer polymerized from acrylic acid or methacrylic acid and is generally referred to under the INCI name of Carbomer. Commercially available Carbomers include Carbopol® polymers 934, 940, 941, 956, 980, and 996 available from Lubrizol Advanced Materials, Inc. In a further aspect, the rheology modifier is selected from a crosslinked copolymer polymerized from a first monomer selected from one or more monomers of acrylic acid, substituted acrylic acid, salts of acrylic acid and salts of substituted acrylic acid and a second monomer selected from one or more C10-C30 alkyl acrylate esters of acrylic acid or methacrylic acid. In one aspect, the monomers can be polymerized in the presence of a steric stabilizer such as disclosed in U.S. Pat. No. 5,288,814 which is herein incorporated by reference. Some of the forgoing polymers are designated under INCI nomenclature as Acrylates/C10-30 Alkyl Acrylate Crosspolymer and are commercially available under the trade names Carbopol® 1342 and 1382, Carbopol® Ultrez 20 and 21, Carbopol® ETD 2020 and Pemulen® TR-1 and TR-2 from Lubrizol Advanced Materials, Inc.

In another aspect, the rheology modifier can be a crosslinked, linear poly (vinyl amide/acrylic acid) copolymer as disclosed in U.S. Pat. No. 7,205,271, the disclosure of which is herein incorporated by reference.

Another class of synthetic rheology modifiers suitable for use in the composition includes hydrophobically modified ASTs, commonly referred to as hydrophobically modified alkali-swellable and alkali-soluble emulsion (HASE) polymers. Typical HASE polymers are free radical addition polymers polymerized from pH sensitive or hydrophilic monomers (e.g., acrylic acid and/or methacrylic acid), hydrophobic monomers (e.g., C1-C30 alkyl esters of acrylic acid and/or methacrylic acid, acrylonitrile, styrene), an “associative monomer”, and an optional crosslinking monomer. The associative monomer comprises an ethylenically unsaturated polymerizable end group, a non-ionic hydrophilic midsection that is terminated by a hydrophobic end group. The non-ionic hydrophilic midsection comprises a polyoxyalkylene group, e.g., polyethylene oxide, polypropylene oxide, or mixtures of polyethylene oxide/polypropylene oxide segments. The terminal hydrophobic end group is typically a C8-C40 aliphatic moiety. Exemplary aliphatic moieties are selected from linear and branched alkyl substituents, linear and branched alkenyl substituents, carbocyclic substituents, aryl substituents, aralkyl substituents, arylalkyl substituents, and alkylaryl substituents. In one aspect, associative monomers can be prepared by the condensation (e.g., esterification or etherification) of a polyethoxylated and/or polypropoxylated aliphatic alcohol (typically containing a branched or unbranched C8-C40 aliphatic moiety) with an ethylenically unsaturated monomer containing a carboxylic acid group (e.g., acrylic acid, methacrylic acid), an unsaturated cyclic anhydride monomer (e.g., maleic anhydride, itaconic anhydride, citraconic anhydride), a monoethylenically unsaturated monoisocyanate (e.g., α,α-dimethyl-m-isopropenyl benzyl isocyanate) or an ethylenically unsaturated monomer containing a hydroxyl group (e.g., vinyl alcohol, allyl alcohol). Polyethoxylated and/or polypropoxylated aliphatic alcohols are ethylene oxide and/or propylene oxide adducts of a monoalcohol containing the C8-C40 aliphatic moiety. Non-limiting examples of alcohols containing a C8-C40 aliphatic moiety are capryl alcohol, iso-octyl alcohol (2-ethyl hexanol), pelargonic alcohol (1-nonanol), decyl alcohol, lauryl alcohol, myristyl alcohol, cetyl alcohol, cetearyl alcohol (mixture of C16-C18 monoalcohols), stearyl alcohol, isostearyl alcohol, elaidyl alcohol, oleyl alcohol, arachidoyl alcohol, behenyl alcohol, lignoceryl alcohol, ceryl alcohol, montanyl alcohol, melissyl alcohol, lacceryl alcohol, geddyl alcohol, and C2-C20 alkyl substituted phenols (e.g., nonyl phenol), and the like.

Exemplary HASE polymers are disclosed in U.S. Pat. Nos. 3,657, 175; 4,384,096; 4,464,524; 4,801,671; and 5,292,843. In addition, an extensive review of HASE polymers is found in Gregory D. Shay, Chapter 25, “Alkali-Swellable and Alkali-Soluble Thickener Technology A Review”, Polymers in Aqueous Media—Performance Through Association, Advances in Chemistry Series 223, J. Edward Glass (ed.), ACS, pp. 457-494, Division Polymeric Materials, Washington, D.C. (1989), the relevant disclosures of which are incorporated herein by reference. Commercially available HASE polymers are sold under the trade names, Aculyn® 22 (INCI Name: Acrylates/Steareth-20 Methacrylate Copolymer), Aculyn® 44 (INCI Name: PEG-150/Decyl Alcohol/SMDI Copolymer), Aculyn 46® (INCI Name: PEG-150/Stearyl Alcohol/SMDI Copolymer), and Aculyn® 88 (INCI Name: Acrylates/Steareth-20 Methacrylate Crosspolymer) from Rohm & Haas, and Novethix™ L-10 (INCI Name: Acrylates/Beheneth-25 Methacrylate Copolymer) from Lubrizol Advanced Materials, Inc.

In another embodiment, acid swellable associative polymers can be used as rheology modifiers. Such polymers generally have cationic and associative characteristics. These polymers are free radical addition polymers polymerized from a monomer mixture comprising an acid sensitive amino substituted hydrophilic monomer (e.g., dialkylamino alkyl (meth)acrylates or (meth)acrylamides), an associative monomer (defined hereinabove), a lower alkyl (meth)acrylate or other free radically polymerizable comonomers selected from hydroxyalkyl esters of (meth)acrylic acid, vinyl and/or allyl ethers of polyethylene glycol, vinyl and/or allyl ethers of polypropylene glycol, vinyl and/or allyl ethers of polyethylene glycol/polypropylene glycol, polyethylene glycol esters of (meth)acrylic acid, polypropylene glycol esters of (meth)acrylic acid, polyethylene glycol/polypropylene glycol esters of (meth)acrylic acid), and combinations thereof. These polymers can optionally be crosslinked. By “acid sensitive” is meant that the amino substituent becomes cationic at low pH values, typically ranging from 0.5 to 6.5. Exemplary acid swellable associative polymers are commercially available under the trade name Structure® Plus (INCI Name: Acrylates/Aminoacrylates/C10-C30 Alkyl PEG-20 Itaconate) from Akzo Nobel, and Carbopol® Aqua CC (INCI Name: Polyacrylates-1 Crosspolymer) from Lubrizol Advanced Materials, Inc. In one aspect, the acid swellable polymer is a copolymer of one or more C1-C5 alkyl esters of (meth)acrylic acid, C1-C4 dialkylamino C1-C6 alkyl methacrylate, PEG/PPG-30/5 allyl ether, PEG 20-25 C10-C30 alkyl ether methacrylate, hydroxy C2-C6 alkyl methacrylate crosslinked with ethylene glycol dimethacrylate. Other useful acid swellable associative polymers are disclosed in U.S. Pat. No. 7,378,479.

Hydrophobically modified alkoxylated methyl glucosides, such as, for example, PEG-120 Methyl Glucose Dioleate, PEG-120 Methyl Glucose Trioleate, and PEG-20 Methyl Glucose Sesquistearate, available from Lubrizol Advanced Materials, Inc., under the trade names, Glucamate® DOE-120, Glucamate™ LT, Glucamate™ VLT and Glucamate™ SSE-20, respectively, are also suitable as rheology modifiers.

Polysaccharides obtained from tree and shrub exudates, such as gum Arabic, gum ghatti, and gum tragacanth, as well as pectin; seaweed extracts, such as alginates and carrageenans (e.g., lambda, kappa, iota, and salts thereof); algae extracts, such as agar; microbial polysaccharides, such as xanthan, gellan, and wellan; cellulose ethers, such as ethylhexylethylcellulose, hydroxybutylmethylcellulose, hydroxyethylmethylcellulose, hydroxypropylmethylcellulose, methylcellulose, carboxymethylcellulose, hydroxyethylcellulose, and hydroxypropylcellulose; polygalactomannans, such as fenugreek gum, cassia gum, locust bean gum, tara gum, and guar gum; starches, such as corn starch, tapioca starch, rice starch, wheat starch, potato starch and sorghum starch can also be employed in the compositions herein as suitable rheology modifiers. A particularly suitable rheology modifier used as thickener is diutan gum.

The rheology modifier(s) can be used alone or in combination and may be present in the composition, on an actives basis, at a total concentration of 0.001-50 wt. %, e.g., at least 0.1 wt. %, or at least 1 wt. %, such as up to 20 wt. %, or up to 10 wt. % %, or up to 3 wt. %, based on the total weight of the composition.

The mascara composition may comprise rheology modifier that is a film forming agent. The film forming agent dissolves in at least one solvent (such as, for example, water and/or organic solvents) in the mascara composition and after the mascara composition is applied to the hair at least one solvent evaporates, absorbs and/or dissipates on the hair, the film forming agent leaves a film on the hair. The use of a film-forming agent improves the wear of the mascara, and can confer transfer-resistance to the mascara. Film forming agents are well known in the art and may be any which is cosmetically acceptable for use around the eye. Examples of useful film-forming agents include natural waxes, polymers such as acrylic acid copolymers, polyethylene polymers, and copolymers of polyvinylpyrrolidone (PVP), ethylene vinyl acetate, dimethicone gum, and resins, such as shellac, polyterpenes, and various silicone resins, e.g., trimethylsiloxysilicate. The film forming agent can be PVP, acrylic acid copolymer and polyurethanes. Suitable film forming agent are polyurethanes such as for example, but not limited to, Avalure™ UR 450 polymer. The film-forming agent can be used in an amount of from about 0.1 to about 50 wt %, or from about 0.5 to about 20.0 wt % based on the total weight of the mascara composition.

Further, the cosmetic compositions may include other active ingredients such as vitamins, minerals, proteins, peptides, fatty acids, antioxidants, anti-inflammatory agents, darkening agents and/or mixtures thereof. Particularly, the cosmetic composition may include other active ingredients for promoting hair growth, and/or preventing hair loss. Suitable, non-limiting examples, in the context of this invention are Growth Oléoactif® [INCI: Helianthus annuus (Sunflower) Seed Oil (and) Polyglyceryl-3 Diisostearate (and) Carthamus tinctorius (Safflower) Flower Extract (and) Hibiscus Sabdariffa Flower Extract] marketed by Hallstar. Widelash™ [INCI: Glycerin, Water (Aqua), Panthenol, Biotinoyl Tripeptide-1] marketed by Sederma; SymPeptide® Xlash [INCI: Glycerin (and) Aqua (and) Myristoyl Pentapeptide-17], SymLash® 1631 [INCI: Pentylene Glycol (and) Isochrysis galbana Extract] marketed by Symrise; Anargy™ [INCI: Water (and) Butylene Glycol (and) Oligopeptide-2 (and) Nicotiana benthamiana Hexapeptide-40 sh-Polypeptide-9 (and) Nicotiana benthamiana Hexapeptide-40 sh-Polypeptide-86] marketed by Lipotrue; Capixyl™ [INCI: Butylene Glycol (and) Aqua (and) Dextran (and) Acetyl Tetrapeptide-3 (and) Trifolium pratense (Clover) Flower Extract] marketed by Lucas Meyer; Nano lashes [INCI: Aqua, Simmondsia chinensis Seed Extract, Polysorbate 20, Hydroxypropyl guar, Sodium Benzoate, Potassium Sorbate] marketed by Nanovetores; SpecPed® SC-MH16 [INCI: Aqua, Glycerin, Myristoyl Hexapeptide-16, Caprylyl Glycol, Ethylhexylglycerin], Myristoyl Pentapeptide-17 [INCI: Myristoyl Pentapeptide-17], SpecPed@ BT1 [INCI: Biotinoyl Tripeptide-1], SpecPed® LashLD [INCI: Biotinoyl Tripeptide-1, Glycerin, Water, Caprylyl Glycol & Ethylhexylglycerin, Panthenol, Trehalose], SpecPed® MP17P [INCI: Myristoyl Pentapeptide-17] marketed by Spec-chem; Procapil™ [INCI: Butylene Glycol (and) Aqua (and) PPG-26-Buteth-26 (and) PEG-40 Hydrogenated Castor Oil (and) Apigenin (and) Oleanolic Acid (and) Biotinoyl Tripeptide-1] marketed by Sederma; AnaGain™ [INCI: Pisum sativum (Pea) Sprout Extract (and) Isomalt (and) Aqua], PhytoCellTec™ Malus domestica Hair [INCI: Malus domestica Fruit Cell Culture Extract (and) Xanthan Gum (and) Glycerin (and) Lecithin (and) Phenoxyethanol (and) Aqua/Water], Santenergy™ [INCI: Bioflavonoids (and) Pentylene Glycol (and) Alcohol (and) Aqua], RootBioTec™ HW [INCI: Ocimum basilicum Hairy Root Culture Extract (and) Alcohol (and) Aqua/Water], RootBioTec™ HO [INCI: Ocimum basilicum Hairy Root Culture Extract (and) Helianthus annuus (Sunflower) Seed Oil (and) Cocos nucifera (Coconut) Oil] marketed by Mibelle; CAPILIA LONGA™ PPF [INCI: Curcuma longa (Turmeric) Callus Culture Conditioned Media (and) Water] marketed by Vytrus Biotech; Cressatine® [INCI: Glycerin (and) Aqua (and) Nasturtium officinale Extract (and) Tropaeolum majus Extract] marketed by Solabia; Alotide™ [INCI: Copper Ascorbyl Phosphate Succinyl Tripeptide-34] marketed by Peptron; BURGEON-UP [INCI: Aqua (and) Alcohol (and) Nasturtium officinale Leaf/Stem Extract] marketed by Ichimaru Pharcos; Capalgin® [INCI: Chondrus crispus Extract] marketed by Exysmol; Dermosaccharides® GY [INCI: Aqua (and) Glycerin (and) Glycogen (and) Phenoxyethanol (and) Methylparaben], Trichogen™ VEG LS 9922 [INCI: Water (and) Panax ginseng Root Extract (and) Arginine (and) Acetyl Tyrosine (and) Arctium majus Root Extract (and) Hydrolyzed Soy Protein (and) Polyquaternium-11 (and) PEG-12 Dimethicone (and) Calcium Pantothenate (and) Zinc Gluconate (and) Niacinamide (and) Ornithine HCl (and) Citrulline (and) Glucosamine HCl (and) Biotin] marketed by BASF; Follicusan™ DP [INCI: Aqua (and) Alcohol Denat. (and) Panthenyl Ethyl Ether (and) Inositol (and) Milk Protein (and) Lactose (and) Acetyl Cysteine (and) Acetyl Methionine (and) Sodium Citrate (and) Citric Acid] marketed by CLR; Hairline® [INCI: Propanediol (and) Water (aqua) (and) Lindera strychnifolia Root Extract] marketed by Greentech; Kerascalp™ [INCI: Propanediol (and) Glycerin (and) Phyllanthus emblica Fruit Extract], Baicapil™ [INCI: Propanediol (and) Water (and) Arginine (and) Lactic Acid (and) Glycine soja (Soybean) Germ Extract (and) Triticum Vulgare (Wheat) Germ Extract (and) Scutellaria baicalensis Root Extract (and) Sodium Benzoate (and) Gluconolactone (and) Calcium Gluconate] marketed by Provital; BIOENERGIZER™ P BG PF [INCI: Aqua/Water, Butylene Glycol, Panthenol, Propylene Glycol, Pelvetia canaliculata Extract, Laminaria digitata Extract] marketed by Seppic; Protectagen™ [INCI: Aqua (and) Glycerin (and) Hydrolyzed Rice Protein] marketed by Ashland; Redensyl® [INCI: Glycerin (and) Aqua (and) Sodium Metabisulfite (and) Larix europaea Wood Extract (and) Glycine (and) Zinc Chloride (and) Camellia sinensis Leaf Extract] marketed by Givaudan; GANOTHER® [INCI: Glycerin (and) Water (and) Ganoderma lucidum (Mushroom) Mycelium Ferment Filtrate] marketed by B&G; Hairgenyl® [INCI: Saccharomyces cerevisiae Extract], Anageline® [INCI: Hydrolyzed Lupine Protein] marketed by Silab; Hairdian™ AP [INCI: Propanediol (and) Thuja orientalis Extract (and) Zingiber officinale (Ginger) Root Extract (and) Trifolium pratense (Clover) Leaf Extract (and) Artemisia argyi Leaf Extract] marketed by Shanghai GREAF Biotech.

The cosmetic composition may also include an ingredient selected from the group consisting of Minoxidil® (Rogaine), Finasteride® (Propecia) and Dutasteride® (Avodart).

### Applications

The invention is based on the finding of surprising properties of a botanical extract from plants of genus Myrothamnus sp. In particular, the properties lend the botanical extract to cosmetic, non-therapeutic applications. Cosmetic, non-therapeutic applications have the aim of improving or maintaining aesthetic appearances and, in particular, this invention is concerned with using the botanical extract to improve or maintain the aesthetic appearance of hair, and, in particular, to improve or maintain the aesthetic appearance of the hair of the eyelashes and/or the eyebrows.

It has been discovered that botanical extracts from plants of genus Myrothamnus are able to stimulate the proliferation of hair follicle cells, thereby stimulating the generation of new hair, and promoting hair growth. Also, it has been discovered that the extracts are able to stimulate the generation of new blood vessels in endothelial cells and stimulate the proliferation of keratinocyte cells, thereby promoting hair growth and strength. Further, it has been discovered that the extract increases the expression of collagen XVII and α6β4 integrin in the hair follicle, thereby increasing hair anchorage. As a result, the botanical extracts of the invention are able to promote hair growth, prevent hair loss; and/or increase the thickness of the hair.

The invention provides for the use of a botanical extract of Myrothamnus sp. for promoting hair growth. The term “promoting hair growth” means stimulating or enhancing hair growth and refers to increasing the density of hair (i.e., the number of hairs per cm² of skin) that is, growing in a given area of the human body. Either optionally or in addition, it also refers to increasing the length of the hair fiber, i.e., increasing the length of the hair in a given area in a period of time as compared to the length without the use of the extract of Myrothamnus sp. for the same period of time.

The density of hair may increase by at least 10%, or least 20%, at least 25%, at least 30%, at least 40%, at least 50%, at least 100%, at least 200% or more when the extract of Myrothamnus sp. is used as compared to the density prior to the use of the extract of Myrothamnus sp.

The length of the hair, where the length is the average length, may increase by at least 10%, or least 20%, at least 25%, at least 30%, at least 40%, at least 50%, at least 100%, at least 200% or more, when the extract of Myrothamnus sp. is used as compared to the average length without the use of the extract of Myrothamnus sp. for the same period of time.

The invention provides for the use of a botanical extract of Myrothamnus sp. for promoting hair growth of the eyelashes and eyebrows.

The invention provides for the use of a botanical extract of Myrothamnus sp. for lengthening of the hair of the eyelashes.

The invention provides for the use of a botanical extract of Myrothamnus sp. for increasing the density of the hair of the eyelashes.

The invention provides for the use of a botanical extract of Myrothamnus sp. for lengthening of the hair of the eyebrows.

The invention provides for the use of a botanical extract of Myrothamnus sp. for increasing the density of hair of the eyebrows.

The invention provides for the use of a botanical extract of Myrothamnus sp. for preventing hair loss. The hair loss may be normal average daily hair loss or hair loss due to aging. The hair loss may be due to mechanical stress, e.g., that caused by styling the hair, or, especially in the case of eyelash and eyebrow hair, due to rubbing with the hands or fingers. In particular, the invention provides for the use of a botanical extract of Myrothamnus sp. for preventing hair loss in the eyelashes and the eyebrows.

The term “prevention” or “preventing”, as used herein, refers to the ability of the botanical extract of Myrothamnus sp. to prevent, delay or hinder the loss of hair. The term “prevention” can be interchangeable with the term “reduction”, i.e., it refers to the ability of the botanical extract to reduce the amount of hair lost. The invention provides for the use of a botanical extract of Myrothamnus sp. for increasing hair anchorage. This results in the hair being more resilient towards mechanical stress, for example.

The invention provides for the of a botanical extract of Myrothamnus sp. for increasing the thickness of hair. The term “thickness” when used to describe hair refers to the average diameter or cross-sectional area of the hair strand. In certain embodiments, the thickness of the hair increases by at least 10%, or least 20%, at least 25%, at least 30%, at least 40%, at least 50%, at least 100%, at least 200% when Myrothamnus extract is used as compared to the thickness prior to the use of Myrothamnus extract.

The botanical extract of Myrothamnus sp. has been shown to be able to induce the proliferation of hair follicle dermal papilla cells and keratinocyte cells, increase blood flow in hair follicles, and increase the synthesis of XVII collagen and α6β4 integrin in hair follicles. Therefore, the invention also refers to the use of a botanical extract of Myrothamnus sp. to promote the hair growth; and/or prevent hair loss and/or increase hair thickness by inducing the proliferation of hair follicle dermal papilla cells; inducing the proliferation of keratinocyte cells and/or associated keratins for the formation of hair; increasing blood flow in hair follicles; increasing the synthesis of XVII collagen and α6β4 integrin in hair follicles.

In one aspect, the invention provides a method of promoting hair growth, and/or preventing hair loss, and/or increasing the thickness of hair comprising administering at least one botanical extract of Myrothamnus sp. as an active ingredient to a subject. The subject is preferably a mammal, and more preferably a human. Preferably, the method is cosmetic and non-therapeutic.

In the above-described uses and methods the botanical extract may be contained in a composition as described above.

The botanical extract is preferably administered by topical application. “Topical application” as used herein denotes putting the botanical extract (or a composition comprising the botanical extract) in contact with the mammalian keratinous tissue such as skin having hair. Particularly, the skin is skin having hair such as the scalp, skin of the face such as skin of the moustache or the beard, eyebrows, and eyelashes. More particularly, the extract or composition is applied to the eyelash line and/or eyebrow, preferably to the eyelash line.

For the above-described methods of the invention, the frequency of application or administration can vary greatly, depending on the needs of each subject, with a recommendation of an application from once a month to ten times a day, preferably from once a week to four times a day, more preferably from three times a week to twice a day, even more preferably once a day.

The invention also extends to the combination of the methods according to the invention with other methods for promoting and/or increasing hair growth, and/or preventing hair loss, and/or increasing the thickness of hair.

The improvement or maintenance of the aesthetic appearance of the hair as afforded by the uses and methods described above, can be applied to both healthy subjects as well as in those which present diseases and/or disorders of the skin, hair, nails and/or mucous membranes. Thus, the method of the invention can be applied to subjects who do not suffer alopecia or subjects who do suffer involutional alopecia, androgenic alopecia, alopecia areata, alopecia universalis, trichotillomania, telogen effluvium or scarring alopecia. Also, the use of the invention is limited to subjects who do not suffer alopecia or subjects who do suffer involutional alopecia, androgenic alopecia, alopecia areata, alopecia universalis, trichotillomania, telogen effluvium or scarring alopecia.

In another aspect, there is provided a botanical extract of Myrothamnus sp. for use as a medicament. In particular, there is provided a botanical extract of Myrothamnus sp. for use in the treatment of a condition related with hair loss or thinning the hair, such as for example, but not limited to, alopecia (including involutional alopecia, androgenic alopecia, alopecia areata, alopecia universalis, trichotillomania, telogen effluvium, scarring alopecia). In another aspect there is provided a method of treating a condition related with hair loss or thinning the hair, such as for example, but not limited to, alopecia (including involutional alopecia, androgenic alopecia, alopecia areata, alopecia universalis, trichotillomania, telogen effluvium, scarring alopecia) comprising administering a therapeutically effective amount of a botanical extract of Myrothamnus sp. to a subject.

The invention is further described in the following non-limiting examples.

### EXAMPLE 1

### Extract Obtained by Conventional Aqueous Extraction

A conventional extraction is carried out on dried Myrothamnus flabellifolia stems with leaves that have been chopped into approximately 1.27-0.22 mm pieces. The plant material is subjected to extraction with water in a ratio of 1:10 (dried plant material: water, w/v). The extraction is performed with water at 45° C. for 4 hours while stirring. The resulting solution is allowed to cool to room temperature (25° C.) and is filtered with 10μm filter. The filtered solution is a botanical extract and this is then diluted with 100% glycerin in a ratio of 1:5 (extract: glycerin, w/w) and the resultant composition is mixed to form a stock solution.

### EXAMPLE 2

### Extract Obtained by Subcritical Water Extraction

A subcritical water extraction (SWE) is carried out on dried Myrothamnus flabellifolia stems with leaves ground to a course powder approximately 1.27-0.25 mm. This powder is subjected to extraction with subcritical water in a ratio of 1:20 (powder: water, w/v). The subcritical water is held at a temperature of 150° C. under a pressure ranging from 10-11 MPa for 1 hour. Subsequently, the resulting solution is allowed to cool to room temperature (25° C.) and is filtered with 10μm filter. The filtered solution is a botanical extract and this is then diluted with glycerin in a ratio of 1:5 (extract: glycerin, w/w). The resultant composition is mixed to form a stock solution.

### EXAMPLE 3

### Compositional Analysis of the Extracts

The stock solutions of Examples 1 and 2 are evaluated for differences in phytochemicals. Total polyphenol content (TPC) is analyzed along with identification of individual phytochemicals.

The total polyphenol content is determined by the Folin-Ciocalteu method. Briefly, 2 mL of extract is mixed with 5 mL of distilled water and then 1 mL of Folin-Ciocalteu reagent is added to the mixture and the mixture is mixed well for 3 min. After 3 min, 5 mL of 10% sodium carbonate solution is added to stop the reaction. After 30 minutes at room temperature, absorbance is measured at 760 and 850 nm using a spectrophotometer. Gallic acid is used as a standard to obtain a calibration curve.

Liquid chromatography with tandem mass spectrometry LC/MS-MS is used for identifying more specific phytochemicals. Ten microliters (10 μL) of extract are mixed with 100μL of 10 mM HCL and 890μL of ultrapure water in a 2 mL LC-MS vial, the samples are diluted 100 times. Injection volume is 2 μL. The mobile phase used for this analysis comprised 0.1% formic acid in acetonitrile (solvent A) and 0.1% of formic acid in ultrapure water (solvent B). The flow rate was 0.2 mL/min at 40° C. column temperature.

The total polyphenol content by the Folin-Ciocalteu method of the aqueous botanical extract of Example 1 was 652 mg GAE/L while that of the stock solution of Example 2 was 763 mg GAE/L. All compounds identified in the stock solutions of Example 1 and Example 2 are listed in Table 1. The ND designation denotes compounds not detected in the sample.

TABLE 1

| | Stock solution | |
|---|---|---|
| | of Example 1 | Stock solution |
| | (conventional | of Example 2 |
| | water extraction) | (SWE Extract) |
| Phytochemical compound | ppm | ppm |
| 3,4-Dihydroxybenzaldehyde | 0.11 | 0.18 |
| 4-Caffeoylquinic acid | 0.51 | 0.53 |
| 4-Hydroxybenzoic acid | 0.23 | 0.16 |
| Afzelin | 0.26 | 0.41 |
| Arbutin | 32.84 | 33.16 |
| Catechin | 0.27 | 0.23 |
| Coniferaldehyde | ND | 0.16 |
| Coumaric acid | 0.80 | 0.29 |
| Dihydrokaempferol | 0.07 | ND |
| Dihydroquercetin | 0.24 | 0.15 |
| Epicatechin | 3.45 | 0.84 |
| Epigallocatechin | 0.57 | 0.14 |
| Ferulic acid | 0.27 | 0.11 |
| Gallic acid | 0.41 | 0.47 |
| Gallocatechin | 0.97 | 0.50 |
| Isoquercitrin | 3.51 | 5.34 |
| Isorhamnetin-3-O-glucoside | ND | 0.47 |
| Kaempferol | 0.06 | 0.13 |
| Kaempferol-3-O-glucoside | 2.84 | 4.85 |
| Kaempferol-3-O-glucuronide | 22.09 | 25.32 |
| Luteolin-7-O-glucuronide | 22.09 | 24.65 |
| Miquelianin | 33.98 | 39.63 |
| Naringenin | ND | 0.31 |
| Naringenin-7-O-glucoside | ND | 0.07 |
| Piceid | ND | 0.20 |
| Quercetin | 0.21 | 0.19 |
| Quercetin-3,4′-O- | 0.09 | 0.09 |
| diglucoside | | |
| Quercetin-3-O-galactoside | 3.87 | 5.66 |
| Quercitrin | 0.83 | 1.15 |
| Syringic acid | 1.52 | 2.25 |
| Trigonelline | 0.23 | 0.23 |
| Tryptamine | 2.73 | 2.71 |
| Vanillic acid | 0.10 | 0.15 |
| Vanillin | 0.41 | 0.40 |
| Arabitol/Ribitol/Xylitol | 4.42 | 3.37 |
| Erythritol/Threitol | 1.18 | 4.60 |
| Inositol | 5.03 | 4.18 |
| Mannitol | 3.86 | 4.01 |
| Sorbitol | 2.44 | 2.45 |
| Fructose | 235.43 | 166.03 |
| Glucose | 319.69 | 240.89 |
| Sucrose | 29.16 | 179.94 |
| Trehalose | 154.70 | 170.50 |
| Total | 891.48 | 927.11 |

### EXAMPLE 4

### In Vitro Proliferation Assay on Hair Follicle Derma Papilla Cells

Myrothamnus flabellifolia stock solutions obtained as described in Example 1 and 2 (‘test products’) are dissolved at 0.5, 1, 2, 2.5 and 5% (v/v) in Papilla Cells Growth medium supplemented with Growth Supplement and filtered through a 0.2 μm syringe filter.

Hair Follicle Dermal Papilla cells from adult human scalp (HFDPC) are grown to a density of 3×10⁴ cells/mL in Papilla Cells Growth Medium at 37° C. in 5% CO₂ humidified air for 5 days. Subsequently, they are split and then seeded for treatment.

HFDPC are seeded in a culture at a density of 45000 cells/mL of 96-well Cell Carrier black plates (PerkinElmer) in culture medium. After 24 hours incubation at 37° C. in 5% CO₂ humidified air, the medium is removed, and fresh culture medium is added with the dilutions of the test products and the vehicle control. Cells treated with medium alone are used as negative control. Fetal Bovine Serum (FBS) was used as positive control.

After 24 hours incubation, live and dead cells are quantified with the LIVE/DEAD® Viability/Cytotoxicity kit (from Thermo Fisher Scientific) that discriminates live from dead cells by simultaneously staining.

Test products are assayed in 3 independent experiments including 4 replicates in each experiment. Table 2 shows the percentage of live and dead HFDPC normalized the total amount of cells (ALL) in the negative control according to the following formula:

$$Live cells ( or dead cells ) ( \% ) = \frac{Amount of live ( or dead cells in test condition )}{Amount of total cells in negative control} \times 100$$

Table 2 also shows the amount of proliferation according to the following formula:

$$Proliferation of live cells ( or dead cells ) ( \% ) = \frac{Amount of live ( or dead cells in test condition )}{Amount of live cells ( or dead cells in negative control ) \times 100}$$

TABLE 2

| | TEST | | | | |
|---|---|---|---|---|---|
| | CONCEN- | | | | Prolif- |
| | TRATION | | | | eration |
| Test product | (%, v/v) | Cells | MEAN | SEM | (mean) |
| Negative | — | ALL | 100 | | |
| control | — | Live | 88.17 | 2.02 | |
| | — | Dead | 11.79 | 0.97 | |
| Positive control | 15 | Live | 115.30 | 5.99 | 27.13 |
| (FBS) | | Dead | 7.29 | 0.67 | −4.50 |
| M. flabellifolia | 1 | Live | 133.10 | 4.96 | 44.93 |
| from Example 1 | | Dead | 1.89 | 0.53 | −9.91 |
| | 2.0 | Live | 128.50 | 1.48 | 40.33 |
| | | Dead | 0.50 | 0.10 | −11.29 |
| | 2.5 | Live | 115.00 | 2.09 | 26.83 |
| | | Dead | 0.48 | 0.10 | −11.31 |
| M. flabellifolia | 1 | Live | 96.69 | 3.18 | 8.52 |
| from Example 2 | | Dead | 5.76 | 0.63 | −6.03 |
| (SWE) | 2.0 | Live | 117.20 | 5.40 | 29.03 |
| | | Dead | 2.03 | 0.33 | −9.76 |
| | 2.5 | Live | 119.70 | 3.70 | 31.53 |
| | | Dead | 0.83 | 0.20 | −10.96 |
| | 5.0 | Live | 124.80 | 2.18 | 36.63 |
| | | Dead | 0.35 | 0.08 | −11.44 |

The results demonstrate that M. flabellifolia extracts increase the number of live cells and reduce the number of dead cells compared to negative control (non-treated cells). Subsequently, the extracts have a proliferative effect on HFDPC.

### EXAMPLE 5

### In Vitro Proliferation Assay on Keratinocyte Cells Keratinocytes express keratins, the most abundant proteins in hair fiber, and contribute to de novo formation of hair.

Myrothamnus flabellifolia stock solutions are dissolved at 0.5, 1, 2, 2.5 and 5% (v/v) in Keratinocyte Growth Medium supplemented with Mix C39016-CaCl₂ solution and filtered through a 0.2 μm syringe filter.

Human Epidermal Keratinocyte cells (HEK) are grown to a density of 4500 cells/cm² in keratinocyte growth medium at 37° C. in 5% CO₂ humidified air during 7 days. After this time, they are split and then seeded for treatment.

HEK are seeded at a density of 70000 cells/mL of culture medium in 96-well Cell Carrier black plates (PerkinElmer) in culture medium. After 24 hours incubation at 37° C. in 5% CO₂ humidified air, medium is removed and fresh culture medium with dilutions of the test products. Cells treated with medium alone are used as negative control. Epidermal Growth Factor (EGF) is used as a positive control. After 24 hours incubation, live and dead cells are quantified with PrestoBlue® Cell Viability Reagent.

HEK quantification is quantified by fluorescence by using cell permeable resazurin-based solution PrestoBlue® from Thermo Fisher Scientific.

Stock solution of Example 1 and 2 are assayed in 3 independent experiments including 4 replicates in each experiment.

### Results

Table 3 shows cell growth values of HEK cells normalized with respect to the negative control.

TABLE 3

| | TEST | | |
|---|---|---|---|
| | CONCEN- | | |
| | TRATION | | |
| TEST ITEM | (%, v/v) | MEAN | SEM |
| Negative control | — | 100.00 | |
| Positive control (EGF) | 10 | 94.41 | 2.44 |
| M. flabellifolia | 0.5 | 114.60 | 5.55 |
| (Example 1) | 1 | 102.30 | 4.81 |
| | 2 | 93.98 | 4.21 |
| | 2.5 | 88.86 | 3.46 |
| | 5 | 61.25 | 2.38 |
| M. flabellifolia | 0.5 | 116.10 | 2.91 |
| (SWE) (Example 2) | 1 | 114.40 | 2.55 |
| | 2 | 102.10 | 3.49 |
| | 2.5 | 86.88 | 3.33 |
| | 5 | 59.11 | 4.43 |

The results demonstrate that the M. flabellifolia extract increases keratinocyte cell proliferation.

### EXAMPLE 6

### In Vitro Study of New Blood Vessel Formation in Human Umbilical Vein Endothelial Cells (HUVEC)

Hair follicles get nourishment from blood flow in the skin having hair. Optimal irrigation of hair follicle increases its nourishment and stimulates the generation of new hair and improves hair growth and strength. Compounds able to generate new blood vessels in the hair follicle are good candidates for the cosmetic treatment of eyelash growth and strength. In vitro assessment of new blood vessels formation involves measurement of the ability of endothelial cells to form three-dimensional tube-like structures. Human Umbilical Vein Endothelial cells (HUVEC) mediate specific connections with extracellular matrix components generating differential forces which induces morphological changes and migration of HUVEC, leading to the formation of tube-like structures. The aim of this study is to evaluate the efficacy of the compound of the invention to generate new blood vessel tubes by means of measuring the ability of endothelial cells to form three-dimensional tube-like structures.

A 96 well plate is pre-coated with cold Extracellular Matrix Solution (Angiogenesis Assay kit, PromoKine) and incubated for 1 hour at 37° C. After the incubation, HUVEC cells are seeded in pre-coated wells at a density of 15,000 cells/well. Immediately, cells are treated with the stock solution of Example 2 dissolved in medium achieving a final well concentration of 0.5% (v/v). Vascular endothelial growth factor (VEGF) is used as positive control at a final well concentration of 5 ng/ml and cells treated with medium alone are used as a basal control. Cells are treated for 4 hours at 37° C. in 5% CO₂ humidified air and then are stained with Staining Dye (Angiogenesis Assay kit, PromoKine) according to the manufacturer's protocol. Briefly, cells are washed and then Staining Dye solution was added. After a 30 minutes incubation at 37° C. in 5% CO₂ humidified air, fluorescence images are captured using Operetta® confocal microscope (PerkinElmer, Inc) and new tubes formation is evaluated by ImageJ software.

New blood vessel tube formation is evaluated by the measurement of different parameters in each image acquired. First, ImageJ detects the constitutive elements of the network such as junctions, segments and branches. Junctions are the nodes which conform a tube bifurcation, a segment is the tube delimited by two junctions and a branch corresponds to an element delimited only by one junction. Afterwards, the software quantifies more complex structures:
Segment length: number of pixels of each segment detected.
Master segment number: number of segments delimited by junctions without branch connection.
Master junction number: number of junctions which link at least three master segments.
Number of meshes: number of elements delimited by segments or master segments.

To consider an increase in new blood vessel tube formation, more than one of the parameters measured needs to be increased.

Results herein are the different measured parameters expressed as percentage normalized by basal condition.

TABLE 4
Mean values of the different measured parameters percentage
respect basal condition on HUVEC cells.

| | | Stock solution |
|---|---|---|
| | VEGF | of Example 2 |
| | 5 ng/ml | 0.5% (v/v) |
| | (Mean ± SEM) | (Mean ± SEM) |
| % of total segment length | 160.1 ± 21.9 | 158.0 ± 22.1 |
| % of number of master segments | 196.5 ± 36.5 | 180.5 ± 36.0 |
| % of number of master junctions | 183.1 ± 31.1 | 173.4 ± 30.3 |
| % of number of meshes | 288.6 ± 75.9 | 298.2 ± 77.8 |

The results confirm that the botanical extract of the invention increases tube-like structures formation respect to basal conditions in HUVEC cells at tested concentration. They also demonstrate that the increase achieved by the botanical extract of the invention is similar to positive control increase.

### EXAMPLE 7

### Ex Vivo Study of Collagen XVII and Integrin Beta-4 Immunostaining in Scalp Explants

Hair anchorage depends upon the adhesion of different cells to the basement membrane zone with hemidesmosomes being the main binding units. Hemidesmosomes are cell-matrix junctions formed by type XVII collagen, two subunits of α6β4 integrin and CD151. Both type XVII collagen and α6β4 integrin interact directly with the basement membrane zone helping to avoid hair loss. Furthermore, collagen XVII proteolysis is related to hair follicle aging. It has been proved that hair follicles miniaturize and often disappear from the skin during aging by means of a miniaturization process, characterized by collagen XVII proteolysis, leading to hair thinning and loss [Matsumura H, et al. “Hair follicle aging is driven by transepidermal elimination of stem cells via COL17A1 proteolysis” Science. 2016 Feb. 5; 351 (6273):aad4395]. Compounds able to increase type XVII collagen and α6β4 integrin are good candidates for the cosmetic treatment of eyelashes and eyebrow aging, strength and loss. The aim of this study was to evaluate the efficacy of the compound of the extract of the invention to improve hair anchoring by means of measuring the increase of collagen XVII and integrin beta-4 (β4) in the different areas of the hair follicle in scalp explants.

Scalp explants are prepared and kept in BEM medium (BIO-EC) at 37° C. in a humid 5% CO₂ atmosphere for 8 days. A 2% (w/w) dilution of the stock solution of Example 2 was applied topically to the explant at day 0, day 4 and day 6. Scalp explants treated with medium alone were used as basal control. At day 8, explants are collected and cut in two parts. Half is fixed in buffered formalin solution and half is frozen at −80° C.

After fixation for 24 hours in buffered formalin, the explants are dehydrated and impregnated with paraffin. Then explants are embedded and 5-μm-thick sections are made using a microtome (Leica). The sections are mounted on histological glass slides. Collagen XVII immunostaining is performed with a monoclonal anti-collagen XVII antibody (Abcam) for 1 h at room temperature using a Vectastain Kit Vector amplifier system avidin/biotin, and revealed by VIP (Vector laboratories, Ref. SK-4600), a substrate of peroxidase giving a violet staining once oxidized. The immunostaining is assessed by microscopical observation and semi-quantified by image analysis.

The frozen explants are cut into 7-μm-thick sections using a cryostat (Leica). Sections are then mounted on silanized glass slides. Integrin beta 4 immunostaining is performed on frozen skin sections with a monoclonal anti-integrin beta 4 antibody (Chemicon) for 1 h at room temperature and revealed by AlexaFluor488 (Life technologies). The nuclei are counterstained by propidium iodide. The immunostaining is performed manually and semi-quantified by image analysis.

The microscopical observations for collagen XVII and integrin beta 4 are imaged with a camera (Olympus) and analyzed with CellSens™ software. Different areas of hair follicle are studied by defining the following regions of interest: Dermal-Epidermal junction, Infundibulum, Upper root sheath, Bulge, Lower root sheath and bulb. In each of these regions, collagen XVII and integrin beta 4 are quantified.

Results herein are the percentage of increase of collagen XVII and integrin beta 4 in hair follicle regions respect basal control.

TABLE 5
Percentage of increase of collagen XVII and integrin beta 4 in
the different regions of hair follicle respect basal control.

| | Collagen | Integrin |
|---|---|---|
| Area | XVII | beta 4 |
| Dermal-Epidermal junction | 28.25% | 55.98%*** |
| Infundibulum | 57.70%* | 187.76%**** |
| Upper root sheath | — | 209.35%**** |
| Bulge | — | 60.20%*** |
| Lower root sheath | 31.40%* | 101.11%** |
| Bulb | 255.97%* | 107.18%* |

(*p < 0.05, **p < 0.01, ***p < 0.001, ****p < 0.0001)

The results demonstrate that the botanical extract of the invention stimulates the hair anchorage by increasing the expression of collagen XVII and integrin beta 4 along the hair follicle.

### EXAMPLE 8

### Mascara for Eyelashes

Mascara for eyelashes according to the invention is prepared as follows:
Ingredients of phase A are mixed under stirring while heating at 85° C. and then dispersed in phase B ingredients. Subsequently, phase D ingredient is added.
Ingredients of phase E are mixed while heating at 90° C. An emulsion is made by slowly adding phase E to the mixture of phases A-D and finally F phase is added.

TABLE 6

| | | | % Material |
|---|---|---|---|
| Phase | Material Name | EU INCI | (w/w) |
| A | WATER | AQUA | 40.05 |
| A | ZEMEA | PROPANEDIOL | 3.00 |
| | PROPANEDIOL | | |
| A | SENSIVA PA40 | PHENYLPROPANOL | 0.80 |
| | (BC043) | PROPANEDIOL | |
| | | CAPRYLYL | |
| | | GLYCOL TOCOPHEROL | |
| B | KELCO-CARE ™ | SPHINGOMONAS FERMENT | 0.15 |
| | Diutan Gum | EXTRACT | |
| C | ARLATONE ™ MAP | POTASSIUM CETYL | 3.00 |
| | 160 K | PHOSPHATE | |
| D | SunPURO ® Black | CI 77499 | 10.00 |
| | Iron Oxide | | |
| E | PERMULGIN ™ 1550 | CERA ALBA | 12.00 |
| E | KAHLWAX 2811 | ORYZA SATIVA | 3.00 |
| | | BRAN CERA | |
| E | KAHLWAX 2442 | COPERNICIA | 2.00 |
| | | CERIFERA WAX | |
| E | KAHLWAX 7302L | SHELLAC WAX | 1.50 |
| E | GLUCATE ™ SS | METHYL GLUCOSE | 4.00 |
| | | SESQUISTEARATE | |
| | | STEARIC ACID | |
| E | SHOREA ROBUSTA | SHOREA ROBUSTA RESIN | 3.00 |
| | RESIN 6723 | OCTYLDODECANOL | |
| E | SCHERCEMOL ™ SHS | ISOSTEARYL | 5.00 |
| | | HYDROXYSTEARATE | |
| E | TOCOPHERYL | TOCOPHERYL ACETATE | 0.50 |
| | ACETATE | | |
| F | Avalure ™ UR | WATER | 10.00 |
| | 450 polymer | PPG-17/IPDI/DMPA | |
| | | COPOLYMER | |
| G | Stock solution | GLYCERIN WATER | 2.00 |
| | (containing Myrothamnus | MYROTHAMNUS FLABELLIFOLIA | |
| | SWE) of Example 2 | LEAF/STEM EXTRACT | |
| | | Total | 100.00 |

### EXAMPLE 9

### Serum for Eyelashes

The ingredients of Phase A are mixed together. Then the ingredient from phase B is added while stirring. The mixture is neutralized with the ingredient of phase C and finally ingredient D is added.

TABLE 7

| | | | % Material |
|---|---|---|---|
| Phase | Material Name | EU INCI | (w/w) |
| A | WATER | AQUA | 90.40 |
| A | ZEMEA | PROPANEDIOL | 5.00 |
| | PROPANEDIOL | | |
| A | SENSIVA ™ | PHENYLPROPANOL | 0.80 |
| | PA40 (BC043) | PROPANEDIOL | |
| | | CAPRYLYL GLYCOL | |
| | | TOCOPHEROL | |
| B | NOVETHIX ™ | AQUA | 1.50 |
| | L-10 | ACRYLATES/ | |
| | | BEHENETH-25 | |
| | | METHACRYLATE | |
| | | COPOLYMER | |
| C | SODIUM HYDROXIDE | AQUA | 0.30 |
| | 20% P/P | SODIUM HYDROXIDE | |
| D | Stock solution | GLYCERIN | 2.00 |
| | (containing | WATER (AQUA) | |
| | Myrothamnus SWE) | MYROTHAMNUS FLABELLIFOLIA | |
| | of Example 2 | LEAF/STEM EXTRACT | |
| | | | 100.00 |

### EXAMPLE 10

### In Vivo Study for the Assessment of Eyelashes Length Increase of Myrothamnus sp. Extract After Long-Term Application in Caucasian Female Volunteers

The study is carried out for 56 days. The panel of volunteers consisted of thirty-one (31) Caucasian female volunteers, aged between 25 and 50 years old. All individuals of the study applied active and placebo products with a half-face design, applying Active serum of Example 9 and mascara of Example 8 on eyelashes of one half-side and Placebo serum and mascara (having the same formulation but not Myrothamnus sp. extract) on the other half-side. Serum is applied on eyelash roots morning and night and mascara is applied only at morning for 56 days. During the treatment period, all volunteers used a cleanser product Eucerin® DermatoCLEAN from Beiesdorf only at night to remove serum and mascara applied at morning and before serum application at night. Subjects served as their own reference and results obtained at time 56 days are compared with those obtained at initial time. Moreover, results obtained with the Active formulations are compared with those obtained with Placebo ones. The efficacy of the compositions is assessed by the measurement of eyelash length.

Lateral macrophotographs from 20° of the face are taken with the software CameraScan™ (Orion Concept) before and after 56 days of product use. Pictures are taken with closed eyes so that upper eyelid eyelashes are lying over a white patch. The acquisitions are taken in parallel polarized light and images are analyzed by ImageJ™ (National Institutes of Health) software. Imaged eyelashes are divided into five sections and the longest eyelash of each section is selected for measurement. Results are shown in Table 8:

TABLE 8
Mean eyelash length increase after 56 days of product application.

| | Mean eyelash |
|---|---|
| | length increase (%) |
| Active serum + mascara | 3.06 * |
| Placebo serum + mascara | 1.16 |

Statistical significance respect initial time:
* p < 0.05 calculated using unpaired Student's t-test vs initial time.

Results demonstrate that, after 56 days of application of the serum and mascara of the invention there is a statistically significant increase of eyelashes mean length compared to initial time. Moreover, the increase of eyelash length is higher with the Active formulations than with Placebo formulations.

### EXAMPLE 12

### In Vivo Study for the Assessment of Eyebrows Density Improvement of Myrothamnus Extract After Long-Term Application in Caucasian Female Volunteers

The study is carried out for 56 days. The panel of volunteers consisted of twenty-eight (28) Caucasian female volunteers, aged between 25 and 50 years old. All individuals of the study applied active serum of Example 9 and placebo serum (having the same ingredients except Myrothamnus sp.) with a half-face design. Serum is applied on eyebrows twice a day (morning and night) during the 56 days of study. Moreover, during the treatment period all volunteers used a cleanser product Eucerin® DermatoCLEAN from Beiesdorf only at night to remove serum applied at morning and before serum application at night. Subjects served as their own reference and results obtained at time 56 days are compared with those obtained at initial time. Moreover, results obtained with the Active serum are compared with those obtained with Placebo ones.

The efficacy of the composition is assessed by eyebrows density measurement. Lateral macrophotographs from 35° of the face are taken with the software CameraScan (Orion Concept) before and after 56 days of product use. Images obtained are analyzed with ImageJ™ software. Eyebrow regions are selected and converted to binary picture. White pixels are counted showing the following results:

TABLE 9
Mean eyebrow density increase after
56 days of product application.

| | Eyebrow density mean increase (%) |
|---|---|
| Active serum | 16.89 ^{Is} |
| Placebo serum | 13.53 ^{Is} |

Statistical significance respect initial time:
^{Is} p < 0.1 calculated using unpaired Student's t test vs initial time.

The results demonstrate that, after 56 days of application of the serum of the invention, there is an increase of eyebrows density compared to initial time.

### EXAMPLE 13

### In Vivo Study for the Assessment of Eyelash Curvature Angle After Long-Term Application in Caucasian Female Volunteers

The study is carried out for 56 days. The panel of volunteers consisted of thirty-one (31) Caucasian female volunteers, aged between 25 and 50 years old are included in the study. All individuals of the study applied active and placebo products with a half-face design, applying Active serum and mascara on eyelashes of one half-side and Placebo serum and mascara on the other half-side. Serum is applied on the roots of the eyelashes once at morning and once at night; and mascara is applied only at morning for 56 days. During the treatment period, all volunteers used a cleanser product (Eucerin® DermatoCLEAN from Beiesdorf) only at night to remove serum and mascara applied at morning and before serum application at night. Active mascara and serum are described in Examples 8 and 9, respectively. Placebo mascara and serum had the same ingredients as the Active mascara and serum except for the Myrothamnus sp. extract. Subjects served as their own reference and results obtained at time 56 days are compared with those obtained at initial time. The efficacy of the compositions is assessed by determining the change of the eyelash curvature angle using the following formula:

$$Percentage ( \% ) of change = \frac{\overline{X} Tx - \overline{X} T 0}{T 0} \times 100$$

X̅ Tx: Average of parameter for all volunteers after x days of product use
X̅ TO: Average of parameter for all volunteers at baseline

A reduction of eyelash curvature is seen as beneficial effect.

Lateral macrophotographs from 90° of the face are taken with the software CameraScan (Orion Concept) before (at initial time) and after 56 days of product use. The acquisitions are taken in parallel polarized light and images are analyzed by ImageJ™ (National Institutes of Health) software. The uppermost eyelash in each image is selected and the curvature angle is measured at the middle point of the eyelash.

TABLE 10
Mean eyelashes' curvature angle change
after 56 days of product application.

| Student's t | Change of eyelash |
|---|---|
| test vs initial time. | curvature angle (%) |
| Active serum + mascara | −3.25 *** |
| Placebo serum + mascara | −0.12 |

Statistical significance respect initial time:
*** p < 0.001 calculated using unpaired

The results demonstrate that, after 56 days of application of the serum and mascara of the invention, there is a statistically significant decrease of the mean curvature angle of the eyelashes compared to initial time.

### EXAMPLE 14

### In Vivo Study for the Assessment of Eyelashes and Eyebrows Hair Loss

The study is carried out for 28 days. Thirty-one (31) Caucasian female volunteers, aged between 25 and 50 years old are included in the study. All individuals in the study applied active and placebo products with a half-face design. For eyelashes, Active serum and mascara on eyelashes of one half-side and Placebo serum and mascara on the other half-side. Serum is applied on the roots of eyelashes once at morning and once at night, and mascara is applied only once at morning for 28 days. Eyebrows are treated with serum using the same pattern of Active and Placebo half design. Serum is applied on eyebrows at morning and at night for 28 days. During the treatment period, all volunteers used a cleanser product only at night to remove serum and mascara applied at morning and before serum application at night. Active serum and mascara are described in Examples 8 and 9. Results obtained with the Active formulations are compared with those obtained with Placebo ones. The efficacy of the compositions is assessed by the total number of hairs lost from eyelashes and eyebrows.

Eyelashes and eyebrows loss is counted daily, and each eyebrow and eyelash are cleansed separately in order to count the number of hairs lost for Active and Placebo products. Results shown in Table 11 correspond to the total sum of all volunteers' number of hairs lost according to each condition and position.

TABLE 11
Total number of hairs lost in eyelashes and
eyebrows for Active and Placebo products.

| | Total number of hairs | Total number of hairs |
|---|---|---|
| | lost from eyelashes | lost from eyebrows |
| Active products | 216 | 246 |
| Placebo products | 233 | 251 |

The results demonstrate that, after 28 days of application of the serum and mascara of the invention there is a decrease in the number of eyelashes and eyebrows hairs lost when active treatment is applied compared to placebo.

1.-5. (canceled)

6. A method of promoting hair growth, and/or delaying or hindering hair loss, and/or increasing the thickness of hair of a subject, the method comprising administering at least one botanical extract of Myrothamnus sp. to said subject, wherein the extract has been obtained using subcritical water as an extraction solvent.

7. A method according to claim 6, wherein the hair is hair of the eyelashes or the eyebrows.

8. A method according to claim 7, wherein said method is selected from the group consisting of: a method of lengthening the hair of the eyelashes, a method of increasing the density of the hair of the eyebrows, and a method of increasing hair anchorage.

9. (canceled)

10. (canceled)

11. A method according to claim 6, wherein the extract is obtained from the aerial parts of Myrothamnus flabellifolia species.

12.-14. (canceled)

15. A method according to claim 6, wherein the botanical extract comprises miquelianin and kaempferol-3-O-glucuronide.

16.-19. (canceled)

20. A method according to cliam 6, wherein the botanical extract is administered by application to the eyelash line and/or to the eyebrow.

21. A method according to claim 6, wherein the hair growth is promoted and/or hair thickness is increased and/or hair loss is delayed or hindered by inducing the proliferation of hair Follicle Dermal Papilla cells;
inducing the proliferation of keratinocyte cells and/or associated keratins for the formation of hair; increasing blood flow in hair follicles; and/or increasing the synthesis of XVII collagen and a6ß4 integrin in hair follicles.

22. A method according to claim 6, wherein the extract is an aqueous extract in a composition which further comprises a water miscible organic solvent.

23. (canceled)

24. A method according to claim 22, wherein the organic water miscible solvent is a polyol.

25. A method according to claim 22, wherein the aqueous extract and the organic miscible solvent are in a ratio from 1:1 to 1:10 by weight.

26. A method according to claim 6, wherein the extract is in a cosmetic composition comprising at least one cosmetically acceptable excipient or ingredient, and wherein the cosmetic composition is selected from the group consisting of a lotion, a shampoo, a serum, and a mascara composition.

27. (canceled)

28. (canceled)

29. A method according to claim 26, wherein the concentration of the extract in the cosmetic composition is from about 0.5 to about 5% (w/w).

30. (canceled)

31. A process for obtaining a botanical extract from Myrothamnus sp. comprising subjecting plant material of Myrothamnus sp. to an extraction with subcritical water, wherein the extraction comprises the steps of:
i) contacting the plant material from Myrothamnus sp. with subcritical water at a temperature of from 120° C. to 220° C., and a pressure suitable to maintain the water in a liquid state for a period of at least 10 min to form an aqueous botanical extract; and
ii) separating the plant material from the aqueous botanical extract.

32. A process according to claim 31, wherein the plant material comprises the stems and/or the leaves of the plant. and wherein the plant material is dried before extraction.

33. (canceled)

34. A process according to claim 32, wherein the ratio of the weight of dried plant material to the volume of water is from 1:10 to 1:40 g/mL.

35. A process according to claim 31, wherein the temperature of the subcritical water is from about 140° C. to about 160° C.

36. A process according to claim 31, comprising the step of mixing the aqueous botanical extract with a water miscible organic solvent.

37. (canceled)

38. A process according to claim 36, wherein a ratio by weight of the aqueous botanical extract to the water miscible organic solvent is from 1:1 to 1:10.

39. An aqueous botanical extract obtained by the process of claim 31.

40.-45. (canceled)

46. A cosmetic composition comprising the aqueous botanical extract of claim 39 and a water-miscible organic solvent.

47. A cosmetic composition according to claim 46, wherein the water-miscible organic solvent comprises a polyol which is present in the composition in an amount of from about 0.01 to 20 wt. %.

48.-72. (canceled)

73. A method according to claim 24, wherein the organic water miscible solvent comprises a polyol selected from the group consisting of C2-C10 aliphatic hydrocarbyl diols and glycerin.
